(19) United States
(12) Patent Application Publication
(10) Pub. No.: US 2012/0221045 A1
(43) Pub. Date: Aug. 30, 2012

(54) NEEDLE UNIT

(76) Inventor: Yu-Chung Chang, Tainan (TW)

(21) Appl. No.: 13/034,723

(22) Filed: Feb. 25, 2011

(51) Int. Cl. A61B 17/06

(52) U.S. Cl. 606/223

(57) ABSTRACT

A needle unit includes an outer cannula and an inner wire passing through a passageway thereof. The outer cannula is straight or curved, and the outer cannula has a piercing end and an open end connected to the passageway. The piercing end has a dull tip. An opening is disposed around the dull tip of the piercing end or at the top of the cannula. The inner wire has a threading part at an end thereof near an opening of the outer cannula, it allows the suture thread passing through the threading part and it is movable inwardly or outwardly via the opening. Therefore, the needle unit can be advantageous to tie the desired vessels or parenchyma of a liver in hepatic resection with less restriction.

### FIELD OF THE INVENTION

This invention relates generally to a surgical needle, and more particularly, to a needle unit applied in hepatic resections.

### BACKGROUND OF THE INVENTION

A successful hepatic resection requires a skillful surgical technique, a safe volume of the remnant liver, and the reduction or elimination of unnecessary ischemic and reperfusion injuries. Skillful surgeons must aim to minimize blood loss during parenchyma division, to secure bleeding or bile leakage from the raw surface of the liver, and to keep remnant major vessels and ducts intact. If all of the above are fulfilled, the postoperative course will be uneventful. Otherwise, cumbersome care becomes necessary.

Hepatic resections always carry a high risk of intraoperative hemorrhage and postoperative hepatic failure. Although mortality rate is decreasing with the refinement of surgical techniques, instruments, and postoperative care, bleeding during parenchyma transection remains a critical hurdle to overcome. In order to reduce blood loss, both inflow and backflow controls are necessary. Several known methods can be applied to block the inflow intermittently or continuously at the level of the hepatoduodenal ligament (e.g. Pringle's maneuver), hilar, or individual segmental branches. Partial blockage of the individual inflow can avoid ischemia and reperfusion injuries on the remnant liver. For the purposes of preventing hepatic failure, the determination of the appropriate ischemic time is critical, especially for a cirrhotic liver. Backflow can be controlled with the inferior vena cava (IVC) exclusion or individual hepatic vein blockage. However, IVC exclusion may compromise the hemodynamic stability; therefore, the use of overloading fluid prior to clamping would add a burden to postoperative recovery.

Hence, it is necessary to provide a surgical needle, so as to facilitate the control of individual inflow and back flow to reduce intraoperative hemorrhage, and to prevent un-necessary ischemic and reperfusion insult to the unrelated area of the liver in order to minimize the risk of postoperative hepatic failure and so on.

### SUMMARY OF THE INVENTION

Accordingly, it is an aspect of the present invention to provide a needle unit, which includes an outer cannula and an inner wire passing through a passageway thereof. The outer cannula is straight or curved, and the outer cannula has a piercing end and an open end connected to the passageway. The piercing end has a dull tip. An opening is disposed around the dull tip of the piercing end or opens at the tip of the cannula. The inner wire has a threading part at an end thereof near an opening of the outer cannula, and the inner wire allows at least a suture thread to pass through the threading part and the inner wire can be moved inwards or outwards via the opening. When the opening is at the top of the cannula, the inner wire may connect with a plug as the piecing end. Therefore, the needle unit can be advantageous to control the desired individual vessels of an organ in partial resection for example, a liver for various kinds of hepatic resections.

According to an embodiment of the present invention, the needle unit may include an outer cannula and an inner wire. The outer cannula has a passageway extending therethrough, an open end with or without a dull tip-piercing end. The outer cannula may be straight or curved. The piercing end is for puncturing soft tissue and organs, and an opening is disposed around the piercing end or at the top of the cannula. The inner wire passes through the passageway and it has a threading part at or near the end of the opening, in which the inner wire is movable inwardly or outwardly via the opening, and the threading part allows at least a suture thread to pass therethrough. When the opening is at the top of the cannula, the inner wire may connect with a plug as the piecing end.

In an example, the outer cannula may be straight or curved with a continuous curvature along its total longitudinal length, in which the curvature is a central angle equal to or greater than 0° and smaller than 180° divided by a length of the outer cannula, and the length is 101% to 250% of a human liver thickness. In another example, the outer cannula may be straight or curved with a tangent chord angle equal to or greater than 0° and smaller than 90° from the open end to the piercing end.

In an example, the opening may be disposed on a side of the outer cannula and adjacently to the dull tip. In another example, the opening may be directly disposed on an end of the dull tip. In the latter example, threading part may be an oval loop, a circle loop, a diamond loop, a U-shaped part or a hook. Alternatively, the threading part may be added with or without a linear protrusion at tops of the oval loop, the circle loop or the diamond loop near the opening of the outer cannula. The piercing end may be a plug disposed on a tip of the linear protrusion for being the dull tip and covering the opening while the needle unit punctures the soft tissue and the organs.

According to a further embodiment of the present invention, the surgical needle unit for hepatic resections may include an outer cannula, an inner wire passing through the passageway, and a handle disposed on the open end of the outer cannula. The handle has a channel connected to the passageway of the outer cannula, so as to allow the inner wire passing through the channel and the passageway.

With application to the aforementioned the needle unit, the outer cannula is straight or curved, and the inner wire allows at least a suture thread passing through the threading part and it is movable inwardly or outwardly via the opening. Therefore, the needle unit can be applied to tie the desired vessel of an organ in partial resections, for example, a liver in hepatic resections.

It is to be understood that both the foregoing general description and the following detailed description are by examples, and are intended to provide further explanation of the invention as claimed.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIGS. 1A and 1B depict cross-sectional diagrams of the needle unit according to embodiments of the present invention.

FIGS. 1C and 1D depict exploded perspective views of the needle units according to embodiments of the present invention.

FIG. 2 depicts a cross-sectional diagram of the needle unit according to another embodiment of the present invention.

FIG. 3 depicts a diagram of the inner wire according to embodiments of the present invention.

FIG. 4 depicts a schematic diagram of the segmental classification of the liver according to Brisbane Terminology of Liver Anatomy and Resections established by International Hepato-Pancreato-Billiary Association (IHPBA) in 2000.

FIGS. 5A and 5B depict sagital sections of the left hepatic lobe according to an embodiment of the present invention.

FIG. 6A depicts a side view of the right hepatic lobe below the rib cage according to another embodiment of the present invention.

FIG. 6B depicts a sagital section of the right hepatic lobe according to another embodiment of the present invention.

### DETAILED DESCRIPTION OF THE PREFERRED EMBODIMENT

Accordingly, the present invention provides a needle unit, which can be applied to effectively and easily tie the desired vessels of an organ in partial resections.

### Structure of Needle Unit

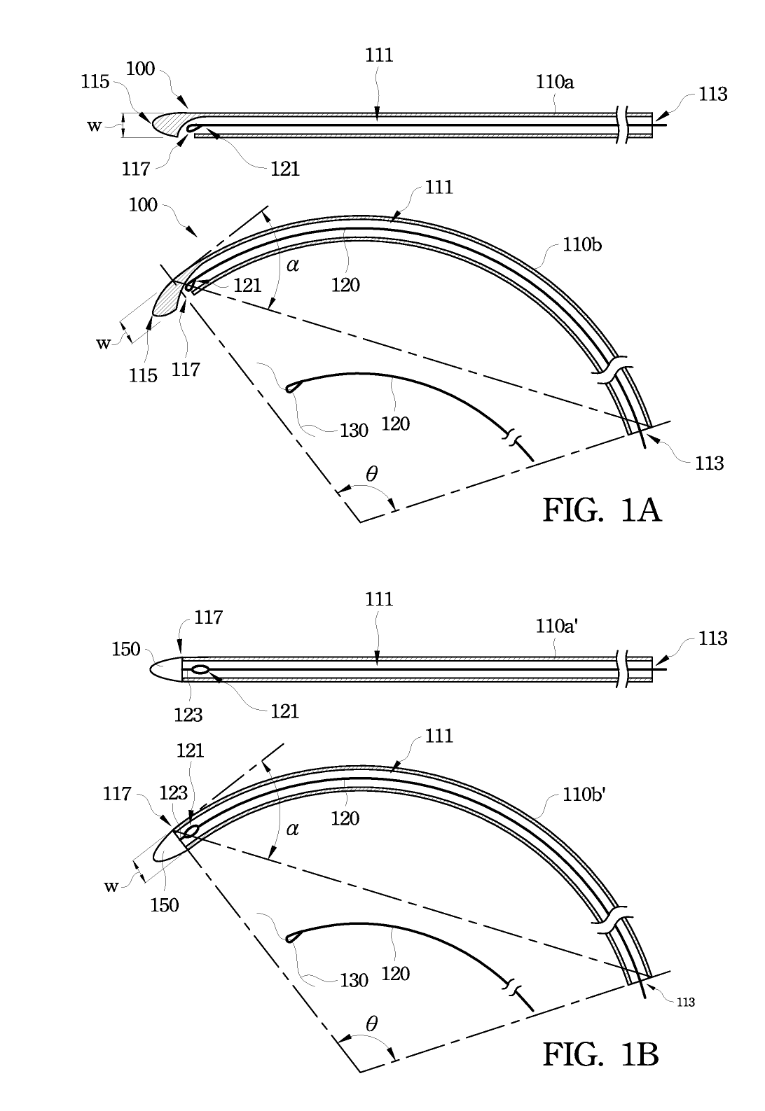

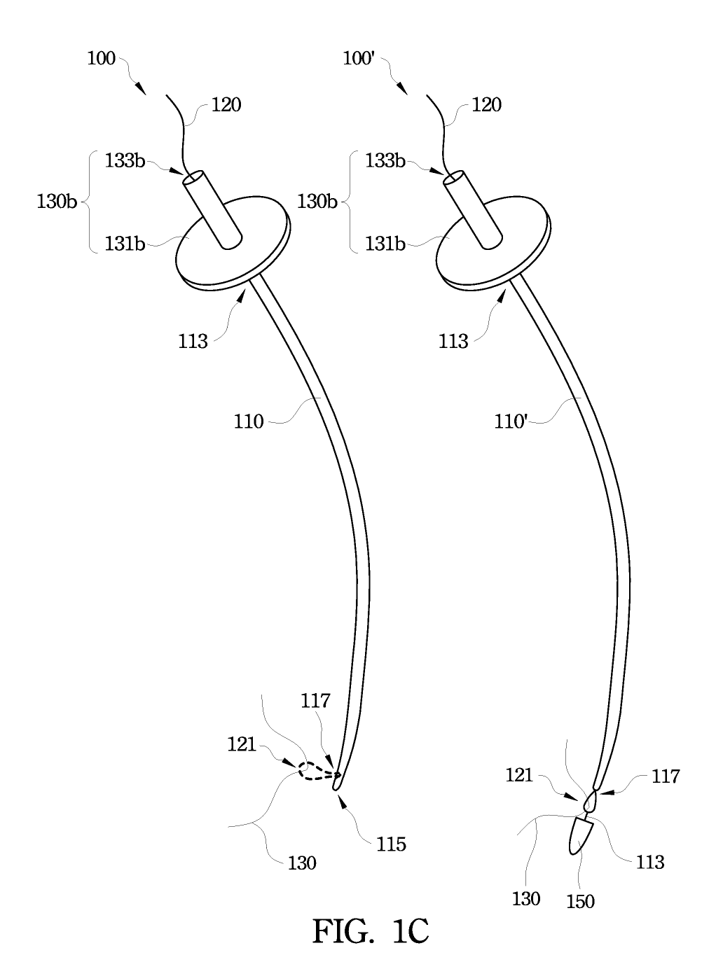

Refer to FIG. 1A. FIG. 1A depicts a cross-sectional diagram of the needle unit according to an embodiment of the present invention. In brief, the needle unit 100 may include an outer cannula (straight outer cannula 110a or curved outer cannula 110b) and an inner wire 120. The outer cannula 110a or 110b has a passageway 111 extending therethrough, an open end 113 connected to the passageway, and a piercing end 115 with a dull tip. In general, the piercing end 115 may be a dull tip for puncturing soft tissue or an organ (for example, liver), and an opening 117 is disposed around the dull tip of the piercing end 115. The inner wire 120 passes through the passageway 111 and it has a threading part 121 (for example, a loop) at an end thereof near the opening 117, in which the inner wire 120 is movable inwardly or outwardly via the opening 117, and the threading part 121 allows at least a suture thread 130 to pass therethrough.

In an example, the opening 117 is disposed on a side of the outer cannula 110a or 110b and adjacent to the dull tip of the piercing end 115, as shown in FIG. 1A. However, in another example, the opening 117 is disposed directly on an end of the outer cannula 110a′ or 110b′, and the outer cannula 110a′ or 110b′ has no dull tip in this example, as shown in FIG. 1B. In this example, threading part 121 may be a loop or a hook. In a case of a loop being the threading part 121, the threading part 121 may have a linear protrusion 123 at its top near the opening 117 of the outer cannula 110a′ or 110b′. The piercing end may be a plug 150 disposed on a tip of the linear protrusion 123 for being the dull tip of FIG. 1A and covering the opening 117 while the needle unit 100′ punctures the soft tissue and the organs. The plug 150 may have a dull tip for puncturing the soft tissue and the organs, and a capping end for covering the opening while the needle unit punctures the soft tissue and the organs. The plug 150 may be made by metal, plastic or other materials appropriately applied in the surgical devices.

In an embodiment, the inner wire 120 may be a metal wire, a plastic wire, or a metal wire coated with a polymer film, for example, a metal wire coated with polytetrafluoroethylene (PTFE).

In an embodiment, the outer cannula 110b can be defined by a tangent chord angle α from the piercing end 115 (above the opening 117) to the open end 113, as shown in FIG. 1A. Alternatively, the outer cannula 110b′ can be also defined by a tangent chord angle α from the opening 117 to the open end 113, as shown in FIG. 1B. The tangent chord angle α is equal to or greater than 0° and smaller than 90° from the opening 117 to the open end 113.

In another embodiment, the outer cannula may be a straight outer cannula 110a (or straight outer cannula 110a′) or curved outer cannula 110b (or curved outer cannula 110b′). In an example, the outer cannula 110b (or curved outer cannula 110b′) may be curved alternatively with a continuous curvature along its total longitudinal length (l), and the curvature is defined by the following equation (I):

$$the curvature of the outer cannula = \frac{1}{r} = \frac{θ}{l} \qquad ( 1 )$$

In the equation (I), the curvature of the outer cannula is directly defined by a reciprocal of its radius (r). The length (l) of the outer cannula 110b (or curved outer cannula 110b′) is defined by multiplying its radius (r) and its central angle (A). In other words, the curvature of the outer cannula 110b is also the central angle (A) divided by the length (l). In an embodiment, the central angle (A) of the outer cannula 110b (or curved outer cannula 110b′) is twice of the tangent chord angle α, or 2α, being equal to or greater than 0 radian and smaller than 3.14 radians.

The length (l) of the outer cannula 110a (or outer cannula 110a′) or the outer cannula 110b (or outer cannula 110b′) may be 101% to 250% (for example, 10 cm to 30 cm) of a human liver thickness. The outer cannula 110a (or outer cannula 110a′) or the outer cannula 110b (or outer cannula 110b′) can be made by metal (for example, metals, metal alloys, stainless steel, or the like) or plastic as known in the prior art, and the outer cannula 110a (or outer cannula 110a′) or the outer cannula 110b (or outer cannula 110b′) has a width w of 14- to 24-gauge (i.e. 2.108 mm to 0.559 mm). Similarly, the outer cannula 110b′ of FIG. 1B can be also alternatively with a continuous curvature along its total longitudinal length (l), and the curvature is also defined by the aformentioned equation (I).

Refer to FIGS. 1C and 1D. FIGS. 1C and 1D depict exploded perspective views of the needle units according to an embodiment of the present invention. The needle unit 100 may include an outer cannula 110 and an inner wire 120, and needle unit 100′ may include an outer cannula 110′ and an inner wire 120 with a plug 150. The outer cannula 110 has a passageway (unshown) extending therethrough, an open end 113 connected to the passageway, and a piercing end 115. The piercing end 115 has a dull tip for puncturing soft tissue or an organ (for example, liver). An opening 117 is disposed around the piercing end 115, being disposed on a side of the outer cannula and closely to the piercing end 115 (the needle unit 100), or directly on an end of the outer cannula 110′ (the needle unit 100′). A threading part 121 at an end of the inner wire 120 is movable inwardly or outwardly from the passageway via the opening 117, and the threading part 121 captures at least a suture thread 130 while it extends outwardly from the opening 117. However, the needle unit of FIGS. 1C and 1D are merely exemplified herein, and it is readily understood that a straight outer cannula may be applied in the needle unit of other embodiments, rather than intending to provide further explanation of the invention as claimed.

In other examples, the threading part 121 of the needle unit 100′ in FIGS. 1C and 1D may have no linear protrusion at tops of the oval loop, the circle loop or the diamond loop near the opening 117 of the outer cannula 110′ (unshown). In the aforementioned examples, those loops 121 of the needle unit 100′ are made by the metal material and a little wider than the opening 117, so as to keep the threading part 121 outside the opening for being the piercing end during puncturing the soft tissue and the organs (unshown).

In an embodiment, a handle, for example, a handle 130b shown in FIG. 1C or a handle 130c shown in FIG. 1D, may be optionally disposed on the open end 113 of the outer cannula 110 or the outer cannula 110′, in which the handle 130b is extended vertically, obliquely on the open end 113 of the outer cannula 110 or the outer cannula 110′, or the handle 130c is extended in the same direction as the outer cannula 110 or the outer cannula 110′. Moreover, the handle 130b shown in FIG. 1C or the handle 130c shown in FIG. 1D respectively has a channel (unshown) in the holding portion 131b or a holding portion 131c to connect an opening 133b or an opening 133c with the passageway 111 of the outer cannula 110 or the outer cannula 110′, so as to allow the inner wire 120 passing from the opening 133b or the opening 133c through the channel and the passageway 111 to the opening 117.

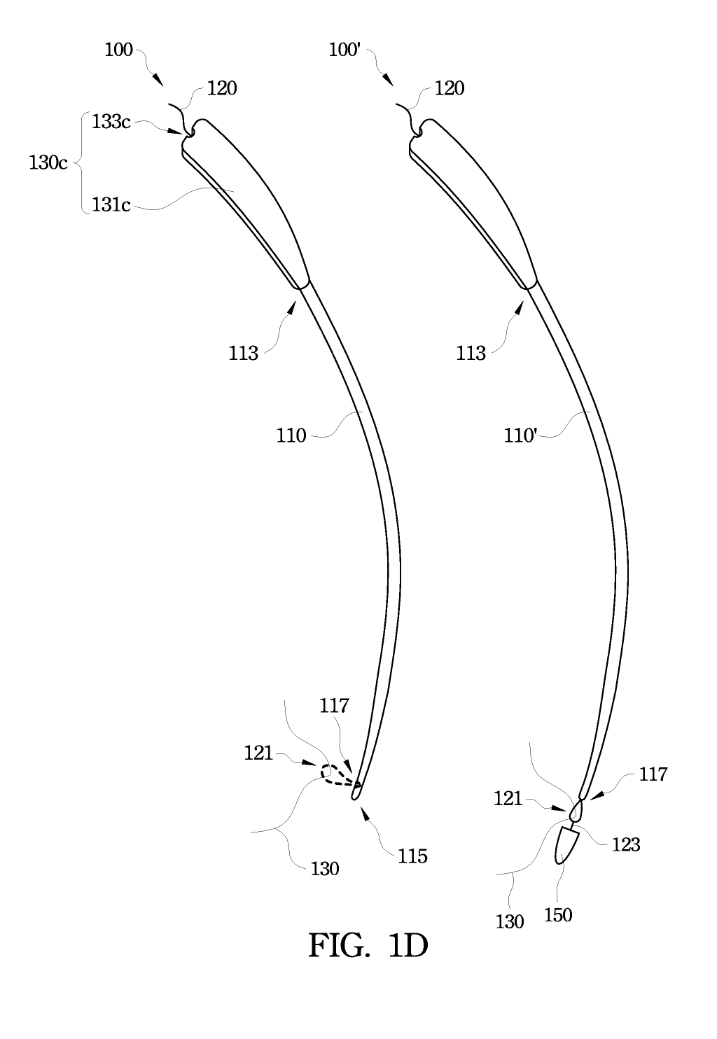

In another embodiment, the outer cannula may be disposed in a hollow tube of a laparoscopic surgical instrument. Refer to FIG. 2. FIG. 2 depicts a cross-sectional diagram of the needle unit according to another embodiment of the present invention. A laparoscopic surgical instrument 140, for example, a trocar instrument commonly available in the prior art, has a hollow tube 141 for accommodating the outer cannula 110 or the outer cannula 110′ to move inwardly or outwardly via the open end 145. In this example, a laparoscopic camera commonly available in the prior art may be disposed separately in another trocar instrument.

In general, the length of the outer cannula 110 or the outer cannula 110′ depends on actual requirements, and it is not intended to limit the scope of the present invention in the length set forth herein. For example, during conventionally open abdomen surgery, the outer cannula 110 or the outer cannula 110′ may have a length corresponding to 101% to 250% (for example, 10 cm to 30 cm) of a human liver thickness when the needle unit 100 or the needle unit 100′ is manually operated. Alternatively, during the laparoscopic operation, when the needle unit 100 or the needle unit 100′ is passed through a trocar port, the outer cannula 110 may have a length longer than the length of the hollow tube 141 shown in FIG. 2, for example, a length of 50 cm approximately.

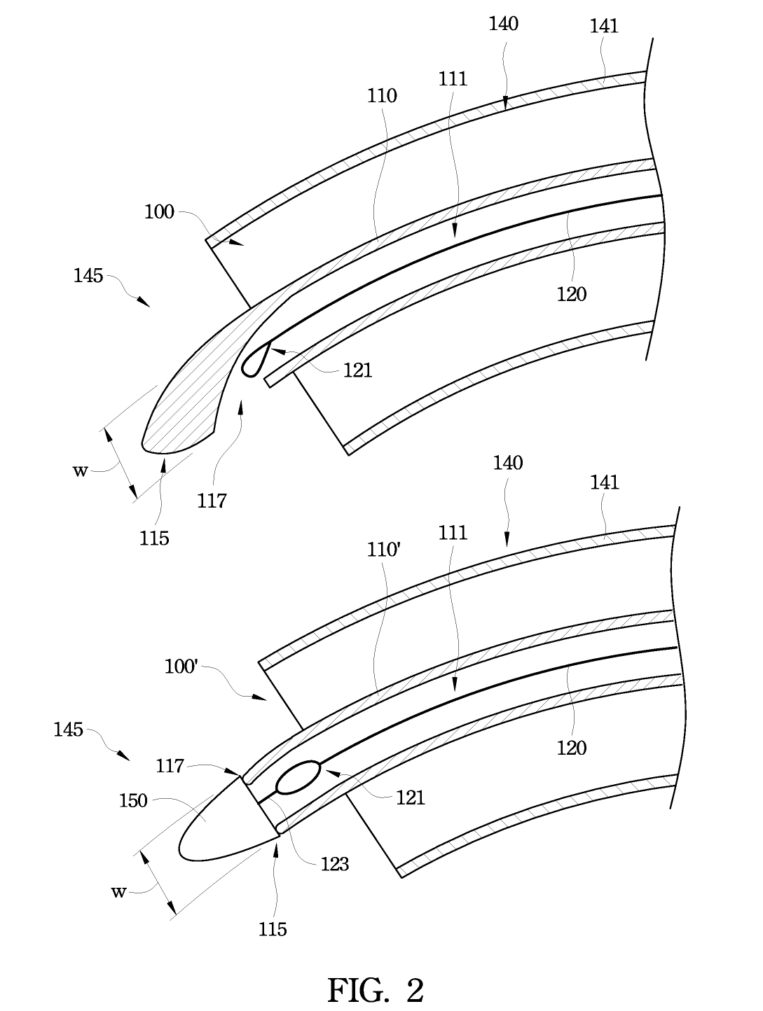

Refer to FIG. 3. FIG. 3 depicts a diagram of the inner wire according to embodiments of the present invention. In an embodiment, the inner wire 120 may be a metal wire, a plastic wire, or a metal wire coated with a polymer film, for example, a metal wire coated with PTFE. The threading part 121 of the inner wire 120 may be various shapes, for example, oval loops (such as the loop 121a and 121b), circle loops (such as the loop 121c and 121d) or diamond loops (such as the loop 121e and 121f). In another embodiment, the threading part 121 of the inner wire 120 may be a U-shaped part 121g. In a still another embodiment, the threading part 121 of the inner wire 120 may be a hook 121h.

Refer to FIG. 3 again. In another embodiment, the threading part 121 may further have a linear protrusion at its top near the opening of the outer cannula (herein referred as the outer cannula 110a or the outer cannula 110b), for example, the protrusion 123b of the loop 121b, the protrusion 123d of the loop 121d, or the protrusion 123f of the loop 121f. The protrusions exemplified as above are facilitated to make the inner wire 120 easier move inwardly or outwardly via the opening. In other examples, the protrusion 123b of the loop 121b, the protrusion 123d of the loop 121d, or the protrusion 123f of the loop 121f may have a plug 150 disposed thereon, respectively, for being the dull tip and covering the opening 117 while the needle unit 100 or the needle unit 100′ punctures the soft tissue and the organs. Alternatively, the inner wire 120 may have a hook 121i and a plug 150 disposed on the end near the opening of the outer cannula 110 (herein referred as the outer cannula 110a or the outer cannula 110b), in which the hook 121i is employed as the threading part 121i, and the plug 150 is employed as the dull tip.

The inner wire 120 allows at least a suture thread 130 to pass through the threading part 121 to tie the desired vessel of the liver in hepatic resections. In an embodiment, the suture thread 130 may be absorbable or non-absorbable material commonly used in the prior art.

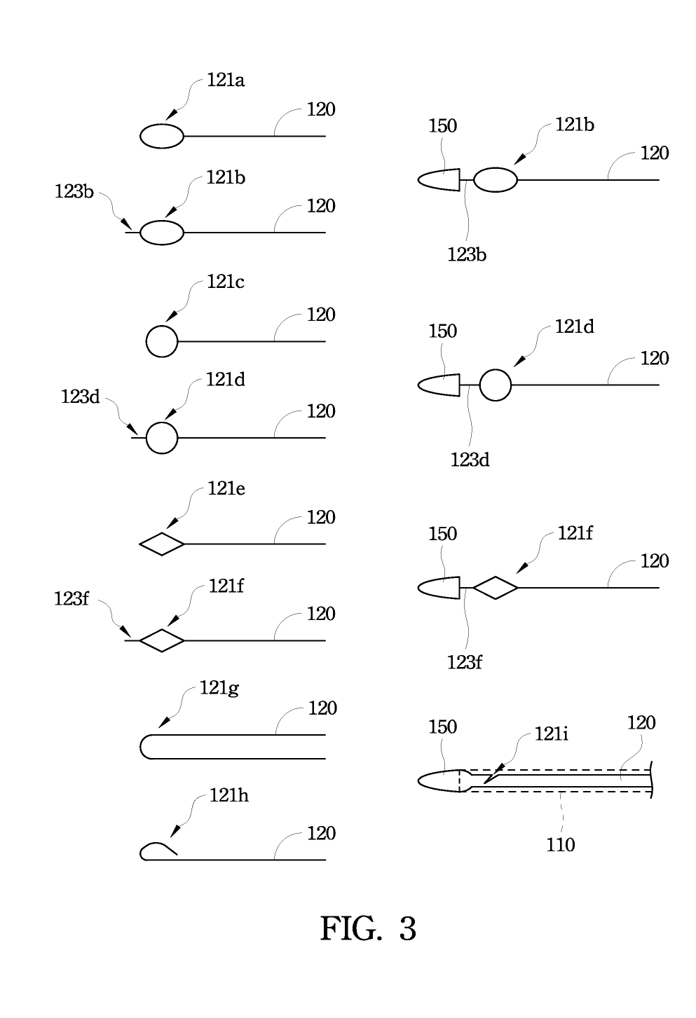

It is worth mentioning that, when the needle unit is curved, it can be applied to tie the desired vessel of an organ more effectively and easily than the straight one. Refer to FIG. 4. FIG. 4 depicts a schematic diagram of the segmental classification of the liver according to Brisbane Terminology of Liver Anatomy and Resections established by International Hepato-Pancreato-Billiary Association (IHPBA) in 2000. The needle unit of the present invention can be applied on the first order, the second-order, the third-order and the fourth-order branches of the Glissonian pedicle in all segments of the liver.

### Application of Needle Unit

During surgery such as hepatic resections, the needle unit can be applied as follows, when taken in conjunction with FIGS. 1A, and 5A-6B. During the hepatic resections, the intraoperative ultrasonography (unshown) or the laparoscopic camera can be properly employed to evaluate the resectability and to determine an appropriate division line. For clarifying the operation of the needle unit in detail, the left hepatic lobe and right hepatic lobe are resected by using the needle unit 100 as exemplified herein. However, it is to be understood that the following description is merely exemplified herein, without intending to provide further explanation of the invention as claimed.

1. Tying the Vessels from Bottom of the Liver, Especially Above the IVC

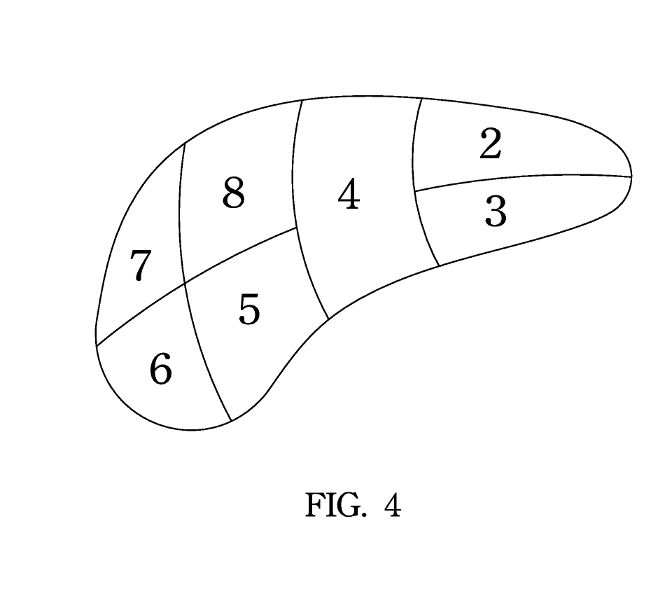

Refer to FIGS. 1A, 5A and 5B. FIGS. 5A and 5B depict sagital sections of the left hepatic lobe according to an embodiment of the present invention. After essentially preoperative preparations, the anterior abdominal wall 509 is lifted. The outer cannula 110b of the needle unit 100 penetrates from the bottom surface 501 (above the inferior venal cava 503) to upper surfaces 505 of liver 500 at the one side of the main supplying vessel 507 (for example, left hepatic Glissonian pedicle) along a path 511, a first end of a suture thread 130 passes through and ties on the threading part 121 of the inner wire 120 of the needle unit 100 while the inner wire 120 moves outwardly via the opening 117 of the outer cannula 110b.

Following, the outer cannula 110b moves back through the upper surface 505 and the bottom surface 503 of liver 500 along the same path 511, bringing along the first end of the suture thread 130 to the liver bottom and leaving a second end of the suture thread 130 on the upper surface 505 of the liver 500. The needle unit 100 then penetrates from the bottom surface 501 to the upper surface 505 at the other side of the main supplying vessel 507 along a path 513, bringing and leaving the first end of the suture thread 130 on the upper surface 505 of the liver 500. The distance between the path 511 and the path 513 on the upper surface is approximately 5 cm. And finally, the first and second ends of the suture 130 on the upper surface 505 of the liver 500 are securely tied as knots 521, so as to control individual inflow or back flow of a target vessel.

In other embodiments, the needle unit 100 is applied repeatedly in either one way as above to make a row of intermittently interlocking sutures 521 along the inner side of the division line of the left hepatic lobe that is resected expectedly, as shown in FIG. 5B. Then, without applying Pringle's maneuver (completely blocking the whole inflows, including common hepatic artery and portal vein outside the liver) or any other procedures for blocking hepatic inflow and backflow, the left hepatic lobe can be divided directly by scissors, electrocautery, or by a Kelly clamp using the clamping and dividing method. Any tubular structures of significant size are suture-ligated for reinforcement. After the aforementioned hepatic resection, it is not necessary to apply fibrin sealant or collagen sheets onto the remained liver surface. Routinely, a large caliber drainage tube can be placed to the dependent portion if necessary.

Any modification of applying suture thread 130 is feasible if two ends of the suture thread 130 can be brought to the same side to tie each other, either on the upper surface 505 or on the bottom surface 501 of the liver 500. Excision of the liver tumor can be performed thereafter in conventional methods. After completing the tumor excision, these knots 521 and suture threads 130 may be left permanently or be cut and removed if restoration of the inflow and outflow of the liver 500 is needed.

The above procedures can be also applied on right hepatic lobectomy or various resections as well when there is little space between the liver upper surface 505 and the abdominal wall 509 to allow applying a straight needle (for example, a needle unit 100 having the outer cannula 110a and the inner wire 120) from the liver surface difficulty.

2. Tying the Vessels (Apart from the IVC) from Liver Surface or Bottom, Especially Under the Condition of a Narrow Space Between the Liver and the Abdominal Wall

Moreover, the needle unit can also be easily to tie the vessels of the liver below a narrow rib cage or a narrow space between the liver and the abdominal wall, where the conventional straight needle is also hardly to reach when the space is limited.

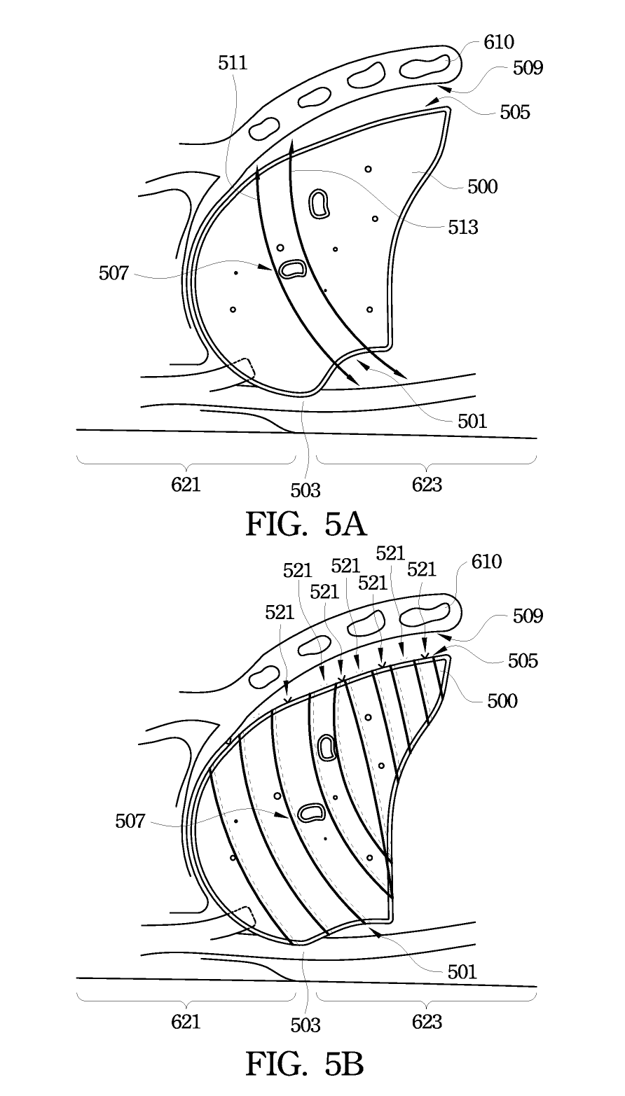

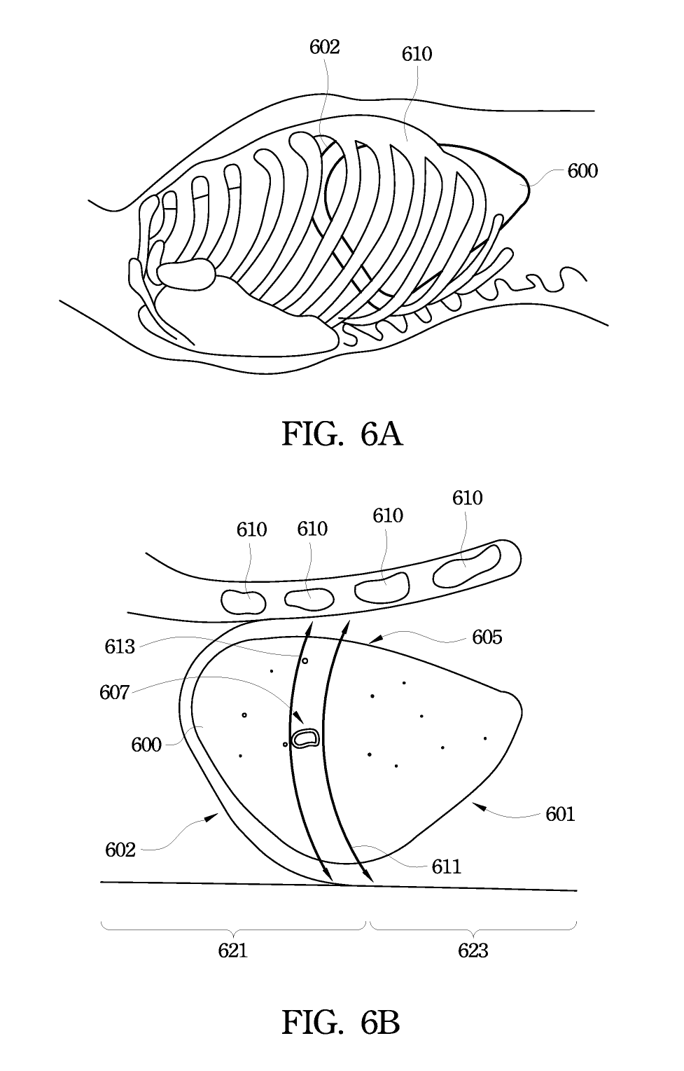

Refer to FIGS. 1A, 6A and 6B. FIG. 6A depicts a side view of the right hepatic lobe below the rib cage 610 and the diaphragm 602 according to another embodiment of the present invention, and FIG. 6B depicts a sagital section of the right hepatic lobe according to another embodiment of the present invention. Similarly, after essentially preoperative preparations, the rib cage 610 is lifted slightly. The outer cannula 110b of the needle unit 100 punctures from the upper surfaces 605 to the bottom surface 601 (apart from the inferior venal cava 503 of FIGS. 5A and 5B) of liver 600 at one side of the main supplying vessel 607 along a path 611, with the outer cannula 110b facing the retroperitoneal abdominal wall 623.

Following, the threading part 121 of the inner wire 120 moves outwardly via the opening 117 of the outer cannula 110b and captures a first end of a suture thread 130. And then, the outer cannula 110b moves back through the bottom surface 603 and the upper surface 605 of liver 600 along the same path 611, leaving the first end of the suture thread 130 on the upper surface 605 of the liver 600. Next, the needle unit 100 penetrates from the upper surfaces 605 to the bottom surface 601 at the other side of the main supplying vessel 607 along a path 613, the threading part 121 of the inner wire 120 moves outwardly via the opening 117 of the outer cannula 110b and captures the second end of the suture thread 130. The distance between the path 611 and the path 613 on the upper surface is approximately 5 cm. And then, the outer cannula 110b moves back through the bottom surface 603 and the upper surface 605 of liver 600 along the same path 613, leaving the second end of the suture thread 130 on the upper surface 605 of the liver 600. Consequently, the first and second ends of the suture 130 are securely tied on the upper surface 605 of the liver 600.

Alternatively, a first end of a suture thread 130 passes through the threading part 121 of the inner wire 120 of the needle unit 100 while the inner wire 120 moves outwardly via the opening 117 of the outer cannula 110b, and then, the inner wire 120 moves inwardly until the tip of the threading part 121 within the opening 117. After the above preparation, the outer cannula 110b of the needle unit 100 penetrates from the bottom surface 601 (either apart or above the inferior venal cava 503) to upper surfaces 605 of liver 600 at one side of the main supplying vessel 607 along a path 611, brings and leaves the first end of the suture thread 130 on the upper surface 605 of the liver 600. Following, the outer cannula 110b moves back through the upper surface 605 and the bottom surface 603 of liver 600 along the same path 611.

After the threading part 121 of the inner wire 120 moves outwardly via the opening 117 of the outer cannula 110b and captures a second end of the suture thread 130, and then the inner wire 120 moves inwardly until the tip of the threading part 121 within the opening 117. After the above preparation, the needle unit 100 penetrates from the bottom surface 601 to the upper surface 605 at the other side of the main supplying vessel 607 along a path 613, brings and leaves the second end of the suture thread 130 on the upper surface 605 of the liver 600. The distance between the path 611 and the path 613 on the upper surface is also approximately 5 cm. And then, the first and second ends of the suture 130 are securely tied on the upper surface 605 of the liver 600, so as to control individual inflow and back flow of a target vessel.

In other embodiments, the needle unit 100 is applied repeatedly in either one way as above to make a row of intermittently interlocking sutures along the inner side of the division line of the hepatic lobe or segment that is resected expectedly, as similarly shown in FIG. 5B. Then, without applying Pringle's maneuver or any other procedures for blocking hepatic inflow and backflow, the hepatic lobe or segment can be divided directly by scissors, electrocautery, or by a Kelly clamp using the clamping and dividing method. Any tubular structures of significant size are suture-ligated for reinforcement. After the aforementioned hepatic resection, it is not necessary to apply fibrin sealant or collagen sheets onto the remained liver surface. Routinely, a large caliber drainage tube can be placed to the dependent portion if necessary.

It is noted that, in comparison with a long straight needle (or a needle unit 100 or a needle unit 100′ having the outer straight cannula 110a and the inner wire 120) for tying the vessels, the needle unit having the outer curved cannula 110b and the inner wire 120 can be sufficiently applied to tie the vessels of the left lobe of the liver in hepatic resections above the IVC, or under the limited space between the liver and the abdominal wall (the abdominal wall 509 of FIGS. 5A, 5B and 6B, or the retroperitoneal abdominal wall 623 of FIGS. 5A, 5B and 6B) of both lobes but without injuring the IVC or pulling up the rib cage excessively or to spare a thoracotomy, where the conventional straight needle is hardly to reach the vessels above the IVC or to apply under the limited space between the liver and the abdominal wall when the liver tumor is huge.

Moreover, the needle unit of the present invention can combine with the long straight needle or a needle unit 100 (or a needle unit 100′) having the outer straight cannula 110a and the inner wire 120 for tying the vessels of the liver in hepatic resections. For example, the vessels above the IVC or a narrow space between the liver and the abdominal wall can be tied by using the needle unit having the outer curved cannula 110b and the inner wire 120, and the vessels far away the IVC can be tied by using the long straight needle or the needle unit 100 having the outer straight cannula 110a and the inner wire 120.

In addition, the needle unit is applied to tie the vessels of the liver as exemplified in the present invention; however, the needle unit can be also applied to tie the desired vessels of other organs in partial resections within the spirit and scope of the appended claims. For example, the needle unit can be applied in the treatment of the female urinary incontinence. In this embodiment, the needle unit can be applied to place a sling tape passed from vagina to connective tissue along both sides of the pubic bone, so as to cradle the urethra and give it support.

According to the above embodiments of the present invention, the aforementioned needle unit of the present invention advantageously includes the outer cannula and the inner wire passing through therein for effectively and easily tying the desired vessels in hepatic resections (Chang's maneuver), especially the ones of the segments 4, 7 and 8, left or middle hepatic veins of the liver in hepatic resections that will be much difficult or sometimes impossible for the straight needle. Therefore, the limitations of the conventional straight needle can be effectively overcome.

As is understood by a person skilled in the art, the foregoing embodiments of the present invention are illustrated of the present invention rather than limiting of the present invention. It is intended to cover various modifications and similar arrangements included within the spirit and scope of the appended claims. Therefore, the scope of which should be accorded to the broadest interpretation so as to encompass all such modifications and similar structure.

What is claimed is:

1. A needle unit, comprising:
an outer cannula, wherein the outer cannula has a passageway extending therethrough, and the outer cannula further comprises:
an open end connected to the passageway; and
a piercing end with a dull tip, wherein the piercing end is for puncturing soft tissue and organs, and an opening is disposed around the dull tip; and
an inner wire passing through the passageway and having a threading part at an end thereof near the opening, wherein the inner wire is movable inwardly or outwardly via the opening, and the threading part allows at least a suture thread to pass therethrough.

2. The needle unit according to claim 1, wherein the outer cannula is straight.

3. The needle unit according to claim 1, wherein the outer cannula is curved with a curvature along its total longitudinal length, the curvature is a central angle equal to or greater than 0° and smaller than 180° divided by a length of the outer cannula, and the length is 101% to 250% of a human liver thickness.

4. The needle unit according to claim 1, wherein the outer cannula having a width of 2.108 mm to 0.559 mm is made by metal or plastic.

5. The needle unit according to claim 1, wherein the inner wire is a metal wire, a plastic wire, or a metal wire coated with a polymer film.

6. The needle unit according to claim 1, wherein the suture thread is absorbable or non-absorbable.

7. The needle unit according to claim 1, wherein the threading part is an oval loop, a circle loop, a diamond loop, a U-shaped part or a hook.

8. The needle unit according to claim 7, wherein the threading part has a linear protrusion at tops of the oval loop, the circle loop or the diamond loop near the opening of the outer cannula.

9. The needle unit according to claim 8, wherein the opening is disposed on a side of the outer cannula and adjacently to the dull tip.

10. The needle unit according to claim 8, wherein the opening is disposed directly on an end of the dull tip.

11. The needle unit according to claim 10, wherein the piercing end has a plug disposed on a tip of the linear protrusion for being the dull tip and covering the opening while the needle unit punctures the soft tissue and the organs.

12. The needle unit according to claim 10, wherein the oval loop, the circle loop or the diamond loop made by metal is a little wider than the opening, so as to keep the threading part outside the opening for being the piercing end during puncturing the soft tissue and the organs.

13. The needle unit according to claim 1, wherein a handle is disposed on the open end of the outer cannula, and the handle has a channel connected to the passageway of the outer cannula, so as to allow the inner wire passing through the channel and the passageway.

14. A needle unit, comprising:
an outer cannula, wherein the outer cannula is straight or curved, the outer cannula has a passageway extending therethrough, and the outer cannula further comprises:
an open end connected to the passageway; and
a piercing end with a dull tip, wherein the piercing end punctures soft tissue and organs, and an opening is disposed around the piercing end; and
an inner wire for passing through the passageway and having a threading part at an end thereof near the opening, wherein the inner wire is movable inwardly or outwardly via the opening, and the threading part allows at least a suture thread to pass therethrough.

15. The needle unit according to claim 14, wherein the outer cannula is curved with a tangent chord angle equal to or greater than 0° and smaller than 90° from the piercing end to the open end.

16. The needle unit according to claim 14, wherein threading part is an oval loop, a circle loop, a diamond loop, a U-shaped part or a hook.

17. The needle unit according to claim 16, wherein the threading part has a linear protrusion at tops of the oval loop, the circle loop or the diamond loop near the opening of the outer cannula.

18. The needle unit according to claim 17, wherein the opening is disposed directly on an end of the dull tip.

19. The needle unit according to claim 18, wherein the piercing end has a plug disposed on a tip of the linear protrusion for being the dull tip and covering the opening while the needle unit punctures the soft tissue and the organs.

20. The needle unit according to claim 19, wherein the oval loop, the circle loop or the diamond loop made by metal is a little wider than the opening, so as to keep the threading part outside the opening for being the piercing end during puncturing the soft tissue and the organs.
